(19) United States
(12) Patent Application Publication
(10) Pub. No.: US 2012/0330255 A1
(43) Pub. Date: Dec. 27, 2012

(54) URINARY CATHETER STRAP FOR SAFE PATIENT WHEELCHAIR TRANSPORT

(75) Inventor: Bonnie Jean Carlin, Gainesville, FL (US)

(73) Assignee: UNIVERSITY OF FLORIDA RESEARCH FOUNDATION, INC., Gainesville, FL (US)

(21) Appl. No.: 13/509,738

(22) PCT Filed: Nov. 10, 2010
(86) PCT No.: PCT/US10/56169

§ 371 Date: May 14, 2012

Related U.S. Application Data
(60) Provisional application No. 61/261,554, filed on Nov. 16, 2009.

(51) Int. Cl. A61G 5/10; A61F 5/44

(52) U.S. Cl. 604/326

(57) ABSTRACT

Disclosed are various embodiments of a urinary catheter strap effective to permit safe patient transport, in one embodiment, the urinary catheter drainage bag support strap has a first end and a second end, at least one aperture located substantially toward the second end of the support strap, at least one receiving means located along the length of the support strap, wherein the first end is capable of receiving an attachment means to removably secure the support strap to a rigid body; wherein the at least one aperture is capable of receiving a catheter drainage bag hanger: and wherein the at least one receiving means is capable of receiving catheter tubing to secure said catheter tubing to said support strap.

### STATEMENT REGARDING FEDERALLY SPONSORED RESEARCH OR DEVELOPMENT

There is no federal government sponsorship associated with this invention.

### TECHNICAL FIELD

The present invention relates generally to medical devices and more specifically to devices that are effective in securing urinary catheters to chairs and wheelchairs to maintain safety and hygiene.

### BACKGROUND ART

Every year, urinary catheters are inserted in more than 5 million patients nationwide. With that, catheter-associated urinary tract infection (CAUTI) is the most common infection contracted by patients. In fact, nearly 40% of all infections acquired in hospitals are urinary tract infections related to catheter use. Although urinary tract infections are relatively easy to treat and rarely extend the length of hospitalization, they increase patient discomfort and almost universally require antibiotic drug therapy, ultimately leading to unnecessary healthcare costs. Moreover, because of increased Medicare and Medicaid scrutiny, CAUTI prevention has become a top priority for hospitals and other healthcare providers.

CAUTI can occur for many reasons, including the patient's lack of hygiene, transmission by health-care personnel handling catheter insertion, and simply a lack of sterility of catheter tubing during extended catheterization. Additionally, one of the primary causes of CAUTI is lack of proper drainage and subsequent urine backflow combined with excessive manipulation of the catheter and its attendant drainage bag during patient transport. Empirical evidence has shown that the failure to have proper tubing and drainage bag placement significantly increases the risk of CAUTI. in order to assure proper drainage and thus reduce the instance of CAUTI, the catheter collection tubing and drainage bag should always remain at an elevation below the patient's bladder and the collection tubing should always remain above the drainage bag.

Wheelchair transport is one of the most common situations where improper catheter tubing and drainage bag placement and excessive manipulation occur. Current practice during wheelchair transport is to simply place the catheter drainage bag on the patient's lap. This practice presents three primary problems: (1) the catheter tubing and drainage bag are at an elevation above the patient's bladder which leads to the aforementioned improper urine drainage; (2) the tubing and drainage bag are susceptible to getting tangled in the wheelchair frame and/or wheels, which leads to excessive manipulation of the catheter, increasing the possibility of infection and injury to the patient, and; (3) it is unsightly and often embarrassing for a patient to have to hold the drainage bag on his/her lap. Thus, there exists a need for an economical and effective device for adequately supporting the catheter tubing and drainage bag during wheelchair transport while also greatly improving aesthetics and ease of use.

### DISCLOSURE OF THE INVENTION

The present invention is device designed to properly support and maintain a catheter drainage bag and tubing during wheelchair transport. In addressing the problems discussed above, the objective of the present invention is threefold: (1) to minimize urine backflow into the patient's bladder during transport; (2) to prevent the catheter tubing from becoming kinked or tangled in the wheels of a wheelchair; and (3) to improve aesthetics over existing designs. In addition to the above features, the device is also designed to be inexpensive, reusable, adjustable to fit various wheelchairs, and simple to use.

FIG. 1 is a perspective view of a preferred embodiment of the present invention. Shown is a support strap 1 which is made out of relatively high-strength synthetic fabric, such as webbing. In a preferred embodiment of the present invention, support strap 1 is approximately 30 inches in length and is thin and flexible. Loop 3 is formed by doubling back the end of the fabric on to itself in a typical fashion. The proximal end of support strap 1 is removably attached to the seatback or seat of a wheelchair or other rigid body by any acceptable attachment means known in the art, such as a clip 4 placed over the proximal end or, alternatively, by a hook (not shown) which is received by opening 6 of loop 3. In another embodiment, the attachment means could be integrated into the strap as a flexible clip or clamp. At least one aperture 5 is located toward the distal end of support strap 1 in order to receive the catheter drainage bag hanger 9 (not shown). In a preferred embodiment, there is a plurality of apertures 5 along the length of support strap 1 to ensure the catheter drainage bag 7 (not shown) is positioned above the floor, depending on the relative dimensions of a given wheelchair or other rigid body, such as a chair.

FIG. 2 is a detailed perspective view of a preferred embodiment of the present invention showing catheter drainage bag 7 secured to support strap 1. Catheter drainage bag hanger 9 (which is integrated into catheter drainage bag 7) is received at the distal end of support strap 1 by aperture 5. Excess catheter tubing 11 may be received by one or more receiving means 10 in order to secure tubing 11 to support strap 1, above catheter drainage bag 7. The receiving means 10 may be a hook-and-loop fastener (i.e. Velcro®) or a resilient plastic clasp placed along the length of support strap 1 or any other like means known in the art sufficient for resiliently retaining tubing 11. In a preferred embodiment, there is sufficient slack in the tubing 11 between retaining member 10 and catheter drainage bag inlet 13 to ensure that the tubing 11 does not become disconnected from inlet 13 during wheelchair transport or patient movement.

FIG. 3 is a perspective view of a preferred embodiment of the present invention, as shown in FIGS. 1 & 2 and described above, attached to a wheelchair 15. Clip 4 is secured to the rear-most edge of the wheelchair seat 17, securing support strap 1 to the wheelchair. Support strap 1 extends across wheelchair seat 17 such that the bottom of the catheter drainage bag 7 terminates approximately 2-8 inches from the floor 20, depending on the relative dimensions of the wheelchair. When a patient is seated in wheelchair 15, support strap 1 is disposed between wheelchair seat 17 and the patient's body, which further stabilizes support strap 1. Accordingly, it is preferable for support strap 1 to be made of a thin and flexible material so as not to cause discomfort for the patient sitting in the wheelchair.

Alternatively, support strap 1 may be dimensioned such that clip 4 can be placed over the top edge of wheelchair seatback 19, securing support strap 1 to the wheelchair. In this embodiment, support strap 1 extends down seatback 19 across wheelchair seat 17 such that the bottom of the catheter drainage bag 7 terminates approximately 2-8 inches from the floor 20, depending on the relative dimensions of the wheelchair. When a patient is seated in wheelchair 15, support strap 1 is disposed between the seating portions of the wheelchair (17 and 19) and the patient's body, which further stabilizes support strap 1. This embodiment permits the support strap 1 to be secured to a wheelchair or other seat that does not have a gap between the seat and seatback.

Alternatively, it is understood that support strap 1 can be secured to any rigid body by clip 4 depending on the application. For example, with the catheter drainage bag 7 attached to support strap 1, an individual assisting the catheterized patient could clip the top of strap either to her own waistband and/or belt or the patient's gown at waist level to keep the bag 7 lower than the tubing; additionally, retaining means 10 ensures that excess catheter tubing does not become tangled.

It is understood that while the embodiments of the present invention above are intended to be utilized in connection with a wheelchair, the present invention is equally capable of being used in connection with any chair or other rigid body where such use is practical.

Further, it will be understood that the preferred embodiments of the present invention have been disclosed by way of example and that other modifications and alterations may occur to those skilled in the art.

### BRIEF DESCRIPTION OF DRAWINGS

FIG. 1 is a top aspect view of one embodiment of the catheter strap of the present invention.

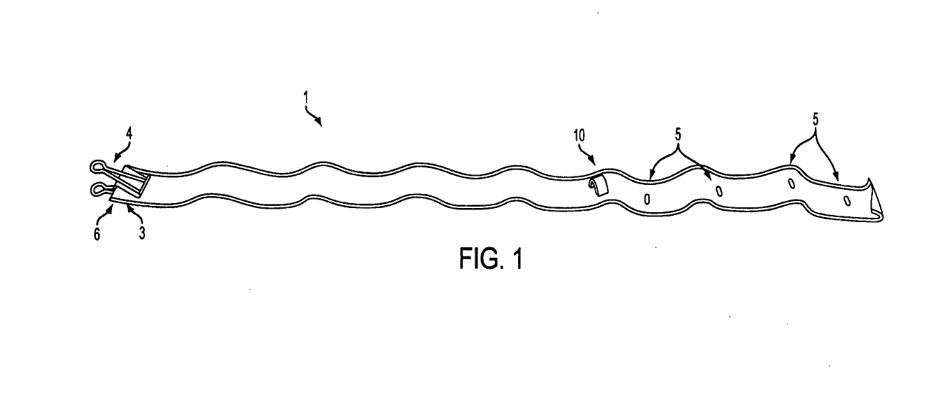

FIG. 2 is another top aspect view of one embodiment of the catheter strap of the present invention, shown with a urinary catheter drainage bag attached thereto.

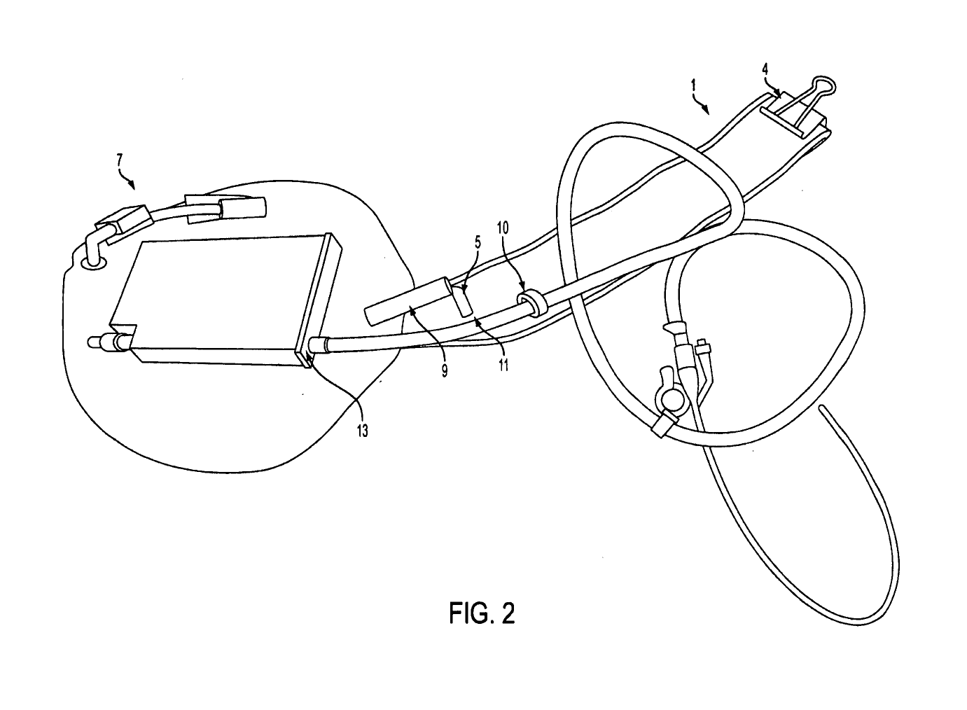

FIG. 3 is a front aspect view of one embodiment of the catheter strap of the present invention, shown as attached to a wheelchair.

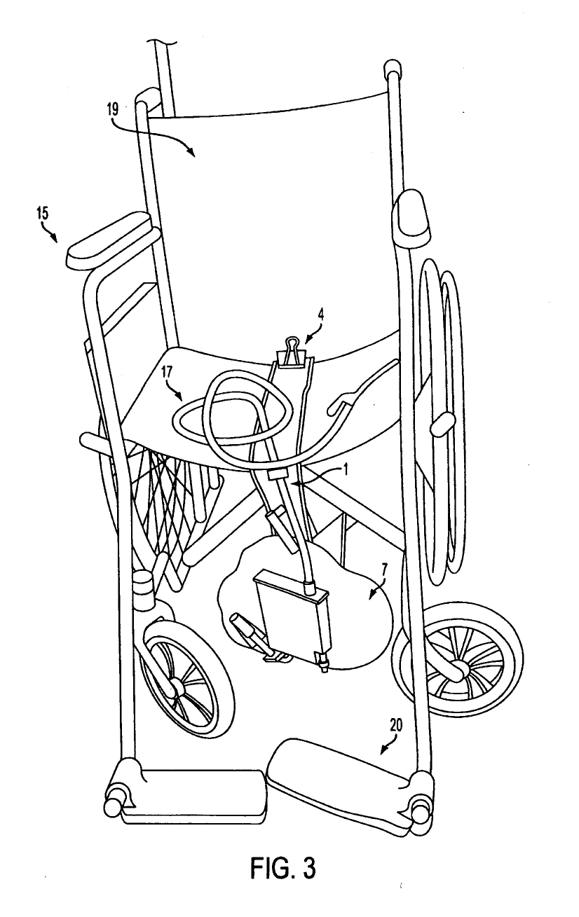

1. A urinary catheter drainage bag support strap, comprising:
a first end and a second end
at least one aperture located substantially toward the second end of said support strap,
at least one receiving means located along the length of said support strap,
wherein said first end is capable of receiving an attachment means to removably secure said support strap to a rigid body;
wherein said at least one aperture is capable of receiving a catheter drainage bag hanger;
wherein said at least one receiving means is capable of receiving catheter tubing to secure said catheter tubing to said support strap;

2. The support strap of claim 1, wherein said rigid body is the seat of a wheelchair.

3. The support strap of claim 2, whereby said support strap is of sufficient length to pass across said seat.

4. The support strap of claim 3, whereby said support strap is disposed between said seat and an individual seated in said wheelchair.

5. The support strap of claim 3, wherein the bottom of said urinary catheter drainage bag terminates at least 2 inches from ground.

6. The support strap of claim 1, wherein said rigid body is the seatback of a wheelchair.

7. The support strap of claim 7, whereby said support strap is of sufficient length to pass down said seatback and across the seating surface of said wheelchair.

8. The support strap of claim 8, whereby said support strap is disposed between said seating surface and an individual seated in said wheelchair.

9. The support strap of claim 8, wherein the bottom of said urinary catheter drainage bag terminates at least 2 inches from ground.

10. The support strap of claim 1, wherein said support strap is thirty (30) inches in length.

11. The support strap of claim 1, wherein said support strap comprises thin, flexible nylon webbing.

12. The support strap of claim 1, wherein said attachment means comprises a clip.

13. The support strap of claim 1, wherein said at least one or more receiving means is a hook-and-loop fastener.

14. The support strap of claim 1, wherein said at least one or more receiving means is a resilient plastic clasp.
